(19) United States
(12) Patent Application Publication
(10) Pub. No.: US 2021/0317113 A1
(43) Pub. Date: Oct. 14, 2021

(54) 1,2,3-TRIAZOLE DERIVATIVES AND USES THEREOF

(71) Applicant: Alxerion Biotech LLC, Cambridge, MA (US)

(72) Inventors: Javier Pedreno, Cambridge, MA (US); Luis Caveda, Port Saint Lucie, FL (US); Jordi Martorell Lopez, Teia (ES); David Sanchez, Barcelona (ES)

(73) Assignee: Alxerion Biotech LLC

(21) Appl. No.: 16/829,597

(22) Filed: Mar. 25, 2020

(51) Int. Cl. C07D 413/14; A61P 35/00; C07D 413/04; C07D 401/04; C07D 249/04

(52) U.S. Cl. CPC C07D 413/14; A61P 35/00; C07D 249/04; C07D 401/04; C07D 413/04

(57) ABSTRACT

Provided herein are 1,2,3-triazole derivatives and methods of use thereof.

### FIELD OF THE INVENTION

The present invention is in the field of medicine and pharmacology. More particularly, the invention relates to hemostasis and compounds useful for the treatment of cancer and disorders resulting from a disruption of hemostasis.

### BACKGROUND OF THE INVENTION

In living organisms, enzymes called proteases are produced to degrade proteins into peptides or amino acids to be used either as an energy source or as building blocks for resynthesize proteins. Proteases also modify cellular environments and facilitate cell migration in connection with wound repair, cancer, ovulation and implantation of the fertilized egg, embryonic morphogenesis, and involution of mammary glands after lactation. In addition, proteases are regulators in process such as inflammation, infection and blood clotting. Proteases act on their natural substrates, proteins and peptides by hydrolyzing one or more peptide bond(s). This process is usually highly specific in the sense that only peptide bonds adjacent to certain amino acids are cleaved. Consequently, most proteolytic enzymes are highly specific for their substrates.

Mammalian serine proteases are one type of protease that may be divided into two families, the trypsins and the subtilisins. The trypsin family includes trypsin, elastase, chymotrypsin, mast cell tryptase, and many of the proteases regulating blood coagulation and fibrinolysis, including thrombin, Factor Xa, plasmin, tissue plasminogen activator (tPA), urokinase plasminogen activator (uPA), and others.

Serine proteases play an important role in fibrinolysis, the degradation of the blood plasma protein, fibrin. Plasminogen is an inactive protein found in blood and is a precursor of plasmin. Plasmin is an enzyme that degrades blood plasma proteins such as fibrin, fibrinogen, Factors V, VIII, IX, XI, and XII. Serine proteases are known to activate plasminogen to plasmin.

The lysis of fibrin clot by specific serine proteases exerts a pivotal role in hemostasis and migration, invasion and proliferation of cancer cells. Serine proteases inhibitors (SERPIN) named antifibrinolytic drugs are synthetic lysine analogues such as EACA (epsilon-amino caproic acid) and TXA (tranexamic acid). Although commonly used to prevent and treat hemorrhages, they all require high doses which are associated with a high frequency of side effects such as headaches, nasal symptoms, and back, abdominal and muscle pain. Despite attempts to reduce these side effects, no other antifibrinolytics exist currently in the market. Thus, what is needed are better methods for stemming bleeding.

Serine proteases also are involved in the breakdown of the extracellular matrix, allowing for cancer invasion and metastasis. It is accomplished by the concerted action of several proteases, including the serine protease plasmin and several matrix metalloproteases. The activity of each of these proteases is regulated by an array of activators, inhibitors and cellular receptors. Thus, the generation of plasmin involves the pro-enzyme plasminogen, the urokinase type plasminogen activator, uPA, and its pro-enzyme, pro-uPA, the uPA inhibitor, PAI-1, the cell surface uPA receptor uPAR, and the plasmin inhibitor a2-anti plasmin.

The plasminogen system also promotes tumor metastasis by several different mechanisms. One of these mechanisms is the uPA and uPAR (uPA receptor) system. The uPA system involves the conversion of plasminogen into plasmin, which plays a key role in cancer invasion and metastasis dissemination by allowing malignant cells to invade the tumor site locally and spread to distant sites. This system includes the serine protease, uPA, membrane-linked receptor uPAR, and two serine protease inhibitors (“SERPINs”), PAI-1 and PAI-2. Thus, plasmin plays a role during multiple steps of cancer invasion and metastasis, by inducing the degradation of a number of ECM proteins and activating certain growth factors leading to aggressive cancers.

Plasminogen receptors also play a role in the proliferation and migration of tumor cells in many cancer types and can serve as prognostic and diagnostic markers. They are involved in mediating colocalization of plasminogen and its activators such as uPA and tPA on cell surfaces and markedly decrease the Km for plasminogen activation. Plasminogen receptors are expressed on the cell surface of most tumors and their expression frequently correlates with cancer diagnosis, survival and prognosis. Notably, they can trigger multiple specific immune responses in cancer patients, highlighting their role as tumor-associated antigens. Cell surface receptors loaded with plasmin, which is protected from inhibitors, play a key role in cancer progression.

Conventional treatment methods for cancer are based on inhibition of proliferation and angiogenesis, and on cytotoxic effects which can also negatively affect normal cells. Thus, what is still needed are improved methods of inhibiting metastatic mechanisms, including the use of inhibitors of uPA/uPAR, plasmin activation, and concurrent metalloproteinases-mediated ECM remodeling to halt cancer progression.

### SUMMARY OF THE INVENTION

It has been discovered that certain 1,2,3-triazole derivatives have SERPIN activity and can inhibit plasminogen activation and the proteolytic activity of plasmin, tPA, and uPA activities. These derivatives can also induce cancer cell death by starvation. These discoveries have been exploited to develop the present disclosure, which, in part, is directed to certain 1,2,3-triazole derivatives and their use in treating disorders resulting in uncontrolled bleeding, cancer, and metastases.

In one aspect, provided herein is a compound of Formula I:

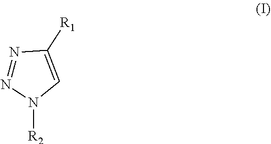

or a pharmaceutically acceptable salt thereof, wherein:

R₁ is selected from the group consisting of hydrogen,

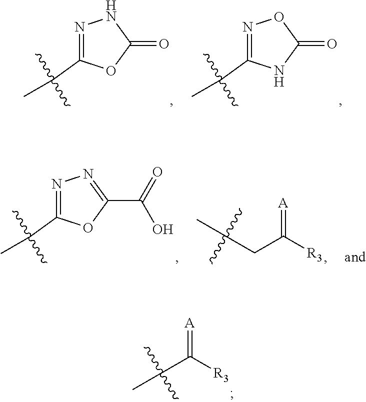

A is independently, at each occurrence, O or NR₄;

R₃ is independently, at each occurrence, NH₂, OH, or NHNH₂;

R₄ is independently, at each occurrence, H or OH;

R₂ is selected from the group consisting of 3-6 membered cycloalkyl, 3-6 membered heterocycloalkyl or C₁-C₆ alkylamine, wherein cycloalkyl, heterocycloalkyl, and alkyl are optionally substituted with one or two R₅; and

R₅ is independently, at each occurrence, selected from the group consisting of NH₂, OH, SH, and halo.

In an embodiment, R₁ is

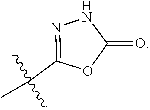 [SMILES: *c1n[nH]c(=O)o1]

In another embodiment, R₁ is

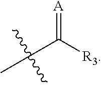

In yet another embodiment, R₁ is

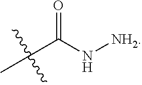 [SMILES: *C(=O)NN]

In still another embodiment, R₁ is

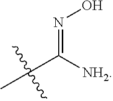 [SMILES: */C(N)=N/O]

In an embodiment, R₁ is

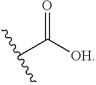 [SMILES: *C(=O)O]

In another embodiment, R₁ is

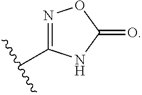 [SMILES: *c1noc(=O)[nH]1]

In yet another embodiment, R₁ is

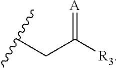

In still another embodiment, R₁ is

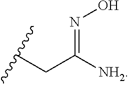 [SMILES: *C/C(N)=N/O]

In another embodiment, R₁ is

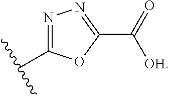 [SMILES: *c1nnc(C(=O)O)o1]

In an embodiment, R₂ is selected from the group consisting of

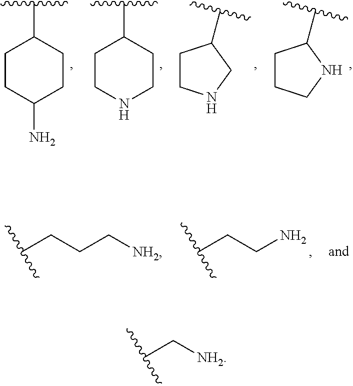

In another embodiment, R₂ is

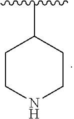 [SMILES: *C1CCNCC1]

In another embodiment, R₂ is

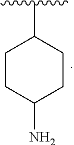 [SMILES: *C1CCC(N)CC1]

In yet another embodiment, R₂ is

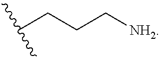 [SMILES: *CCCN]

In another embodiment, R₂ is

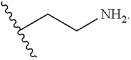 [SMILES: *CCN]

In an embodiment, the compound of Formula I is selected from the group consisting of:

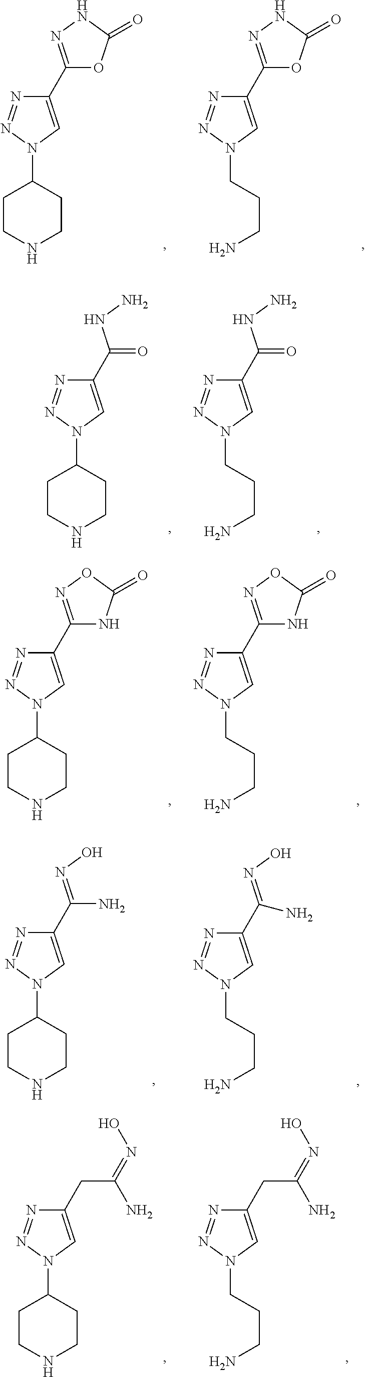

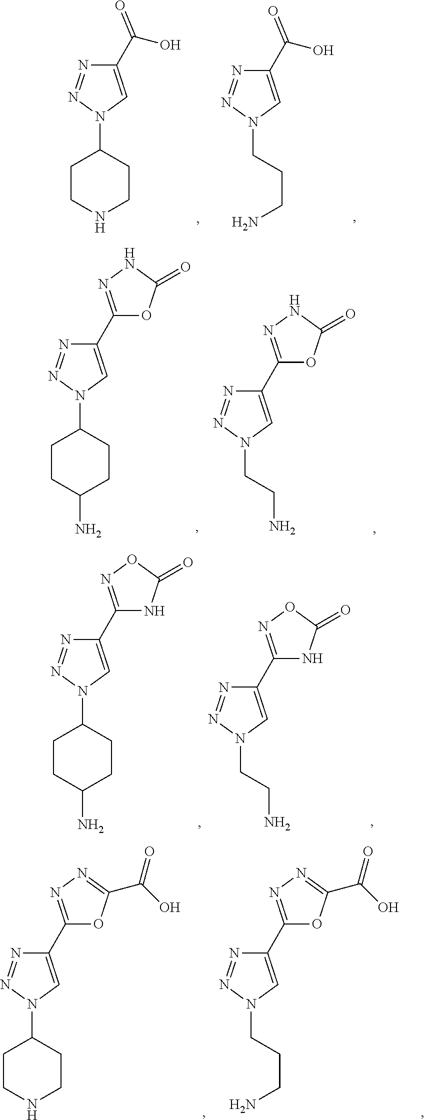

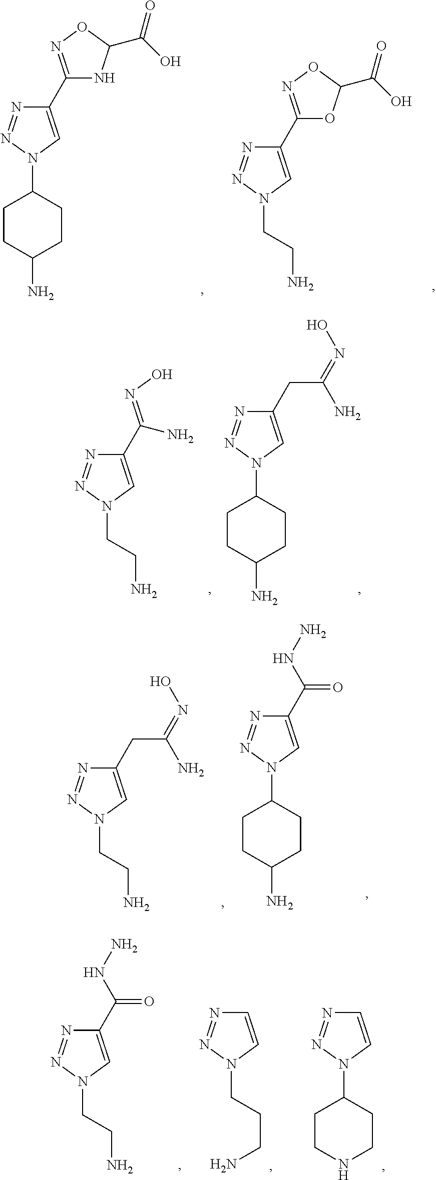

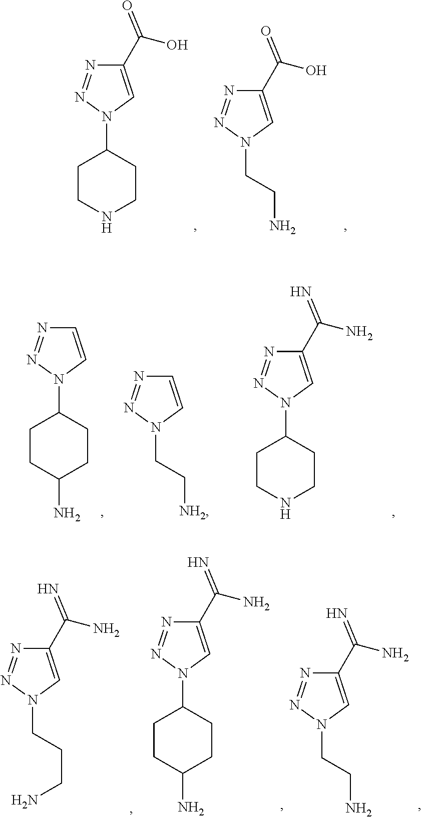

and a pharmaceutically acceptable salt thereof.

In yet another aspect, the disclosure provides a pharmaceutical formulation comprising at least one of the 1,2,3-triazole derivatives described above and a pharmaceutically acceptable carrier.

In one embodiment, the pharmaceutical formulation further comprises another a therapeutic agent for stemming bleeding. In certain embodiments, that agent is different than the 1,2,3-triazole derivative in the formulation.

In another embodiment, the pharmaceutical formulation further comprises another a therapeutic agent for treating cancer or metastasis. In certain embodiments, that agent is different than the 1,2,3-triazole compound in the formulation.

In still another aspect, the disclosure provides a method of treating bleeding in a subject, comprising administering to the subject a therapeutically effective amount of a formulation described herein comprising at least one 1,2,3-triazole derivative. In some embodiments, the formulation comprises another agent which stems bleeding. In other embodiments, the method further comprises administering a therapeutically effective amount of a formulation comprising another anti-bleeding agent and a pharmaceutically acceptable carrier. In certain embodiments, the anti-bleeding agent in the formulation is different than the a 1,2,3-triazole derivative in the formulation administered.

In some embodiments, the bleeding disorders that are treated by the pharmaceutical formulations and methods according to the disclosure include, but are not limited to, spontaneous bleeding, cardiac surgery (i.e., cardiopulmonary bypass), liver transplant, following therapeutic thrombolysis, congenital anti-plasmin deficiency, acquired anti-plasmin deficiency, hemophilia A and B, quantitative and qualitative platelet dysfunction, genitourinary bleeding, upper and lower urinary tract, dysfunctional uterine bleeding (essential menorrhagia and menorrhagia associated with intrauterine device), gastrointestinal bleeding (upper by varices, gastritis, ulcers, and lower by inflammatory bowel disease), mucous membrane bleedings for recurrent epistaxis or for excessive bleeding following tonsillectomy, traumatic hyperemia, trauma, general surgery, orthopedic surgery or cancer. The methods are also useful to treat bleeding due to lack of coagulation factors, V, VII, VIII, or IX, or lack of von Willebrand's factor. In addition, the methods according to the disclosure can be used to treat bleeding as the result of administration of an anticoagulant treatment.

In yet another aspect, the disclosure provides a method of treating a cancer or metastasis in a subject, comprising administering to the subject a therapeutically effective amount of a formulation described herein comprising at least one 1,2,3-triazole derivative. In some embodiments, the formulation comprises another anti-cancer agent. In other embodiments, the method further comprises administering a therapeutically effective amount of a formulation comprising another anti-cancer agent and a pharmaceutically acceptable carrier. In certain embodiments, the anti-cancer agent in the formulation is different than the a 1,2,3-triazole derivative in the formulation administered.

In some embodiments, the anti-cancer agent is an alkylating agent (including, but not limited to, cisplatin, chlorambucil, and procarbazine), an antimetabolite (including, but not limit to, methotrexate, cytarabine, and gemcitabine), an anti-microtubule agent (including, but not limited to, vinblastine and paclitaxel), a topoisomerase inhibitor (including, but not limited to, etoposide and doxorubicin) and/or a cytotoxic agent (including, but not limited to, bleomycin).

Another aspect is directed to the use of a 1,2,3-triazole derivative according to the disclosure, or a salt thereof, for the manufacture of a medicament for treating a bleeding disorder. In some embodiments, the medicament is for reducing clotting time, in other embodiments, the medicament is for prolonging the clot lysis time. In yet other embodiments, the medicament is for increasing clot strength. In yet other embodiments, the medicament is formulated for topical, oral, or intravenous or intramuscular injection administration.

Yet another aspect is directed to the use of a 1,2,3-triazole derivative according to the disclosure, or a salt thereof, for the manufacture of a medicament for treating a cancer or metastasis. In some embodiments, the medicament is for inhibiting the growth, reducing the size, or inhibiting the metastasis of, a cancer. In other embodiments, the medicament is formulated for topical, oral, or intravenous or intramuscular injection administration.

The disclosure provides another aspect directed to a method of inhibiting the serine protease activity of an enzyme selected from the group consisting of tissue plasminogen activator, urokinase plasminogen activator, or plasmin, comprising contacting the enzyme with a 1,2,3-triazole compound as described herein.

### DESCRIPTION OF THE DRAWING

The foregoing and other objects of the present disclosure, the various features thereof, as well as the disclosure itself may be more fully understood from the following description, when read together with the accompanying drawings in which:

FIG. 1 is a diagrammatic representation of the synthesis of a representative 1,2,3-derivative according to the disclosure;

FIG. 2A is a graphic representation of the anti-fibrinolytic activity of Derivative 5 in μM;

FIG. 2B is a graphic representation of the anti-fibrinolytic activity of Derivative 1 in μM;

FIG. 2C is a graphic representation of the anti-fibrinolytic activity of Derivative 7 in μM;

FIG. 3 is a graphic representation of the migration rate of NSLC cells by cell count in the presence of increasing concentrations of FBS (control), 20% plasma, Derivative 1 (LT16), anti-metastatic drug Tranexamic Acid (TNA, and anti-metastatic drug Caproic acid (CA);

FIG. 4A is a photographic representation of the effect of the viability of NSLC grown in the presence of FBS for 7 days, as measured by Trypan Blue staining of NSLC cells in vitro;

FIG. 4B is a photographic representation of the effect of the viability of NSLC grown in the presence of 20% plasma for 7 days, as measured by Trypan Blue staining of NSLC cells in vitro;

FIG. 4C is a photographic representation of the effect of the viability of NSLC grown in the presence of 20% plasma+200 μM Derivative 1 (LT16) for 7 days, as measured by Trypan Blue staining of NSLC cells in vitro;

FIG. 4D is a photographic representation of the effect of the viability of NSLC grown in the presence of FBS for 15 days, as measured by Trypan Blue staining of NSLC cells in vitro;

FIG. 4E is a photographic representation of the effect of the viability of NSLC grown in the presence of 20% plasma for 15 days, as measured by Trypan Blue staining of NSLC cells in vitro; and

FIG. 4F is a photographic representation of the effect of the viability of NSLC grown in the presence of 20% plasma+200 μM Derivative 1 (LT16) for 15 days, as measured by Trypan Blue staining of NSLC cells in vitro.

### DESCRIPTION

The disclosures of these patents, patent applications, and publications in their entireties are hereby incorporated by reference into this application in order to more fully describe the state of the art as known to those skilled therein as of the date of the invention described and claimed herein. The instant disclosure will govern in the instance that there is any inconsistency between the patents, patent applications, and publications and this disclosure.

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including 5%, ±1%, and ±0.1% from the specified value, as such variations are appropriate to perform the disclosed methods.

The term “treat,” “treated,” “treating,” or “treatment” includes the diminishment or alleviation of at least one symptom associated or caused by the state, disorder or disease being treated. In certain embodiments, the treatment comprises bringing into contact with an infection an effective amount of an anti-infective formulation of the disclosure for conditions related to infections.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, but are not limited to, livestock and pets, such as ovine, bovine, porcine, canine, feline and marine mammals.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the CSIC compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Nonlimiting examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids.

The term “cycloalkyl,” employed alone or in combination with other terms, refers to a non-aromatic hydrocarbon ring system (monocyclic, bicyclic or polycyclic), including cyclized alkyl and alkenyl groups. The term “Cn-m cycloalkyl” refers to a cycloalkyl that has n to m ring member carbon atoms. Cycloalkyl groups can include mono- or polycyclic (e.g., having 2, 3 or 4 fused rings) groups and spirocycles. Cycloalkyl groups can have 3, 4, 5, 6 or 7 ring-forming carbons (C₃₋₇). In some embodiments, the cycloalkyl group has 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like. A cycloalkyl group containing a fused aromatic ring can be attached through any ring-forming atom including a ring-forming atom of the fused aromatic ring. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, bicyclo[1.1.1]pentanyl, bicyclo[2.1.1]hexanyl, and the like. In some embodiments, the cycloalkyl group is cyclopropyl, cyclobutyl, cyclopentyl, or cyclohexyl.

The term “heterocycloalkyl,” employed alone or in combination with other terms, refers to a non-aromatic ring or ring system, which may optionally contain one or more alkenylene groups as part of the ring structure, which has at least one heteroatom ring member independently selected from nitrogen, sulfur, oxygen and phosphorus, and which has 4-10 ring members, 4-7 ring members, or 4-6 ring members. Included within the term “heterocycloalkyl” are monocyclic 4-, 5-, 6- and 7-membered heterocycloalkyl groups. Heterocycloalkyl groups can include mono- or bicyclic (e.g., having two fused or bridged rings) or spirocyclic ring systems. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (e.g., C(O), S(O), C(S) or S(O)₂, N-oxide etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring.

The term “alkyl” employed alone or in combination with other terms, refers to a saturated hydrocarbon group that may be straight-chained or branched. The term “Cn-m alkyl,” refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, i-butyl, tert-butyl, isobutyl, sec-butyl; higher homologs such as 2-methyl-1-butyl, n-pentyl, 3-pentyl, ii-hexyl, 1,2,2-trimethylpropyl and the like.

The term “alkylamine” employed alone or in combination with other terms, refers to an alkyl group as defined herein further substituted with an amine group (NH₂), wherein the amine can be further substituted once or twice with alkyl, i.e., NH(alkyl) and N(alkyl)₂.

The present disclosure provides novel 1,2,3-triazole derivatives and salts thereof. Triazoles are heterocyclic organic compounds containing a core of a five-membered ring with three nitrogen atoms and two carbon atoms. One isomeric form of triazole is 1,2,3-triazole.

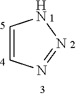

Both isomeric forms of 1,2,3-triazole derivatives are of importance in medicinal chemistry and can be used for the synthesis of numerous heterocyclic compounds with different biological activities such as antiviral (anti-HIV-1), antibacterial, antifungal, antimalarial, antitubercular, anti-obesity, antihypertension, anticonvulsant, anxiolytic, antidepressant, local anaesthetic, anti-inflammatory, antihistaminic, and anticancer activities. 1,2,3-triazoles, they are resistant to oxidation, reduction, and hydrolysis in both acidic ad basic conditions due to their higher aromatic stabilization. Their active participation in hydrogen bond formation, dipole-dipole and pi stacking interactions enhance their binding ability to different biological targets. In addition, a 1,2,3-triazole core also provides diverse pharmacophore properties.

As used herein, the term “1,2,3-triazole derivative” or “derivative” refers to a compound having a 1,2,3-triazole core as described above.

The 1,2,3-triazole derivatives according to the disclosure have the structure of Formula I

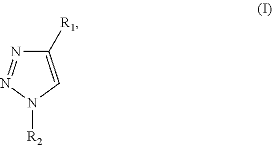

or a pharmaceutically acceptable salt thereof, wherein:

R₁ is selected from the group consisting of hydrogen,

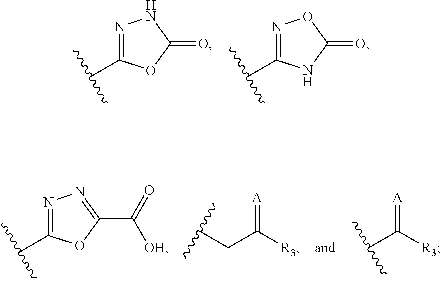

A is independently, at each occurrence, O or NR₄;

R₃ is independently, at each occurrence, NH₂, OH, or NHNH₂;

R₄ is independently, at each occurrence, H or OH;

R₂ is selected from the group consisting of 3-6 membered cycloalkyl, 3-6 membered heterocycloalkyl or C₁-C₆ alkylamine, wherein cycloalkyl, heterocycloalkyl, and alkyl are optionally substituted with one or two R₅; and

R₅ is independently, at each occurrence, selected from the group consisting of NH₂, OH, SH, and halo.

In some 1,2,3-triazole derivatives, R₁ is

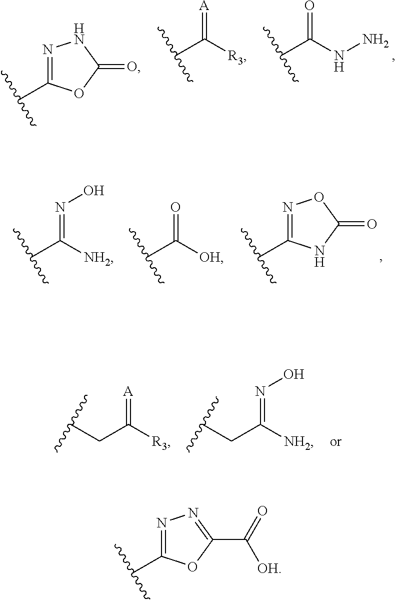

In some 1,2,3-triazole derivatives, R₂ is

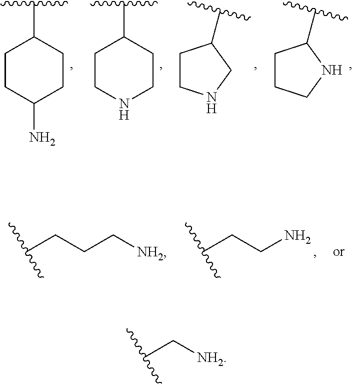

Representative examples of the 1,2,3-triazole derivatives are shown below in Table 1.

TABLE 1

| | |
|---|---|
| 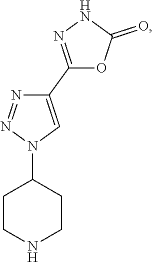 [SMILES: O=c1[nH]nc(-c2cn(C3CCNCC3)nn2)o1] | 1 |
| 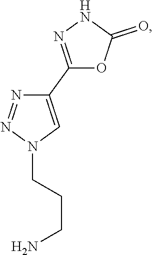 [SMILES: NCCCn1cc(-c2n[nH]c(=O)o2)nn1] | 2 |
| 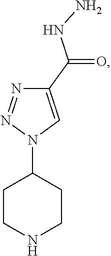 [SMILES: NNC(=O)c1cn(C2CCNCC2)nn1] | 3 |
| 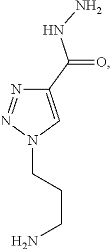 [SMILES: NCCCn1cc(C(=O)NN)nn1] | 4 |
| 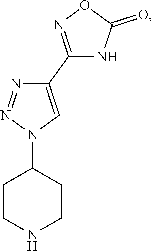 [SMILES: O=c1[nH]c(-c2cn(C3CCNCC3)nn2)no1] | 5 |
| 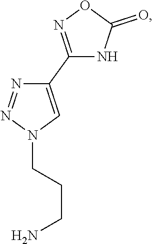 [SMILES: NCCCn1cc(-c2noc(=O)[nH]2)nn1] | 6 |
| 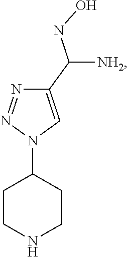 [SMILES: NC(NO)c1cn(C2CCNCC2)nn1] | 7 |
| 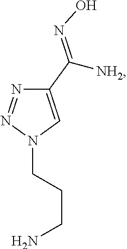 [SMILES: NCCCn1cc(/C(N)=N/O)nn1] | 8 |
| 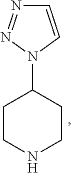 [SMILES: c1cn(C2CCNCC2)nn1] | 9 |
| 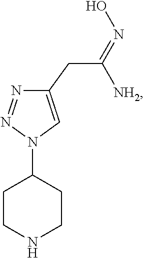 [SMILES: N/C(Cc1cn(C2CCNCC2)nn1)=N/O] | 10 |
| 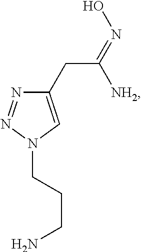 [SMILES: NCCCn1cc(C/C(N)=N\O)nn1] | 11 |
| 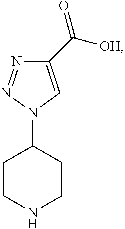 [SMILES: O=C(O)c1cn(C2CCNCC2)nn1] | 12 |
| 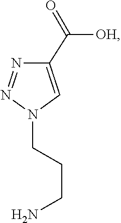 [SMILES: NCCCn1cc(C(=O)O)nn1] | 13 |
| 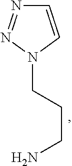 | 14 |
| 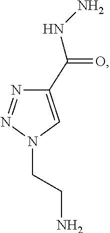 [SMILES: NCCn1cc(C(=O)NN)nn1] | 15 |
| 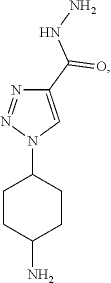 [SMILES: NNC(=O)c1cn(C2CCC(N)CC2)nn1] | 16 |
| 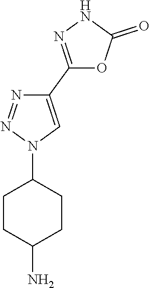 [SMILES: NC1CCC(n2cc(-c3n[nH]c(=O)o3)nn2)CC1] | 17 |
| 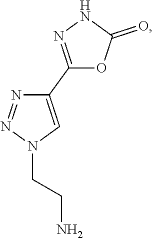 [SMILES: NCCn1cc(-c2n[nH]c(=O)o2)nn1] | 18 |
| 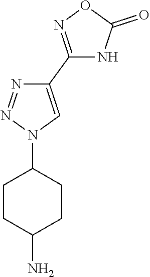 [SMILES: NC1CCC(n2cc(-c3noc(=O)[nH]3)nn2)CC1] | 19 |
| 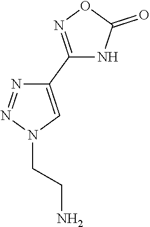 [SMILES: NCCn1cc(-c2noc(=O)[nH]2)nn1] | 20 |
| 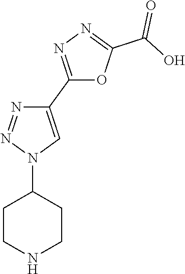 [SMILES: O=C(O)c1nnc(-c2cn(C3CCNCC3)nn2)o1] | 21 |
| 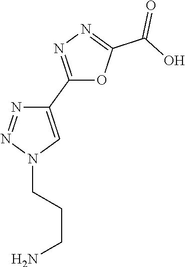 [SMILES: NCCCn1cc(-c2nnc(C(=O)O)o2)nn1] | 22 |
| 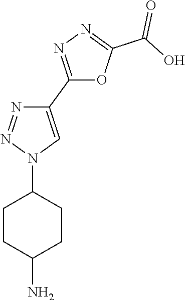 [SMILES: NC1CCC(n2cc(-c3nnc(C(=O)O)o3)nn2)CC1] | 23 |
| 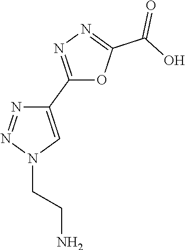 [SMILES: NCCn1cc(-c2nnc(C(=O)O)o2)nn1] | 24 |
| 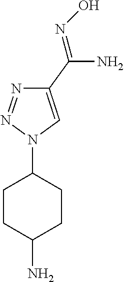 [SMILES: N/C(=N\O)c1cn(C2CCC(N)CC2)nn1] | 25 |
| 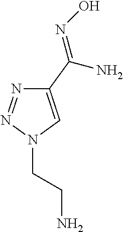 [SMILES: NCCn1cc(/C(N)=N/O)nn1] | 26 |
| 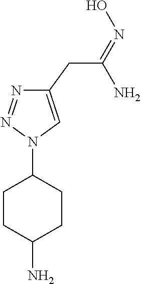 [SMILES: N/C(Cc1cn(C2CCC(N)CC2)nn1)=N/O] | 27 |
| 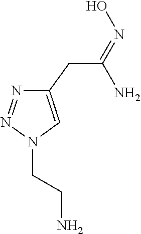 [SMILES: NCCn1cc(C/C(N)=N\O)nn1] | 28 |
| 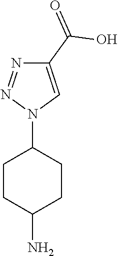 [SMILES: NC1CCC(n2cc(C(=O)O)nn2)CC1] | 29 |
| 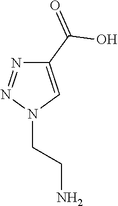 [SMILES: NCCn1cc(C(=O)O)nn1] | 30 |
| 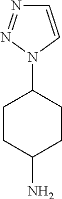 [SMILES: NC1CCC(n2ccnn2)CC1] | 31 |
| 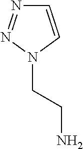 | 32 |
| 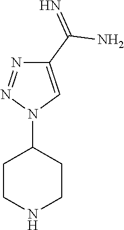 [SMILES: N=C(N)c1cn(C2CCNCC2)nn1] | 33 |
| 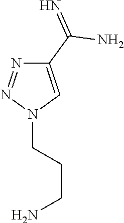 [SMILES: N=C(N)c1cn(CCCN)nn1] | 34 |
| 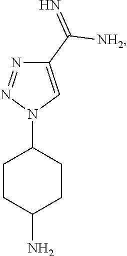 [SMILES: N=C(N)c1cn(C2CCC(N)CC2)nn1] | 35 |
| 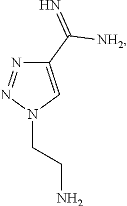 [SMILES: N=C(N)c1cn(CCN)nn1] | 36 |

The salts of the compounds of Formula (I) are pharmaceutically acceptable salts. However, other salts may, however, be useful in the preparation of the compounds according to the disclosure or of their pharmaceutically acceptable salts. Suitable pharmaceutically salts of the compounds include acid addition salts which can be formed by mixing a solution of a pharmaceutically acceptable acid such as hydrochloric acid, sulfuric acid, methanesulphonic acid, fumaric acid, maleic acid, succinic acid, acetic acid, benzoic acid, oxalic acid, citric acid, tartaric acid, carbonic acid or phosphoric acid. Furthermore, where the compounds of the disclosure carry an acidic moiety, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts.

### Synthesis of 1, 2, 3-Triazole Derivatives

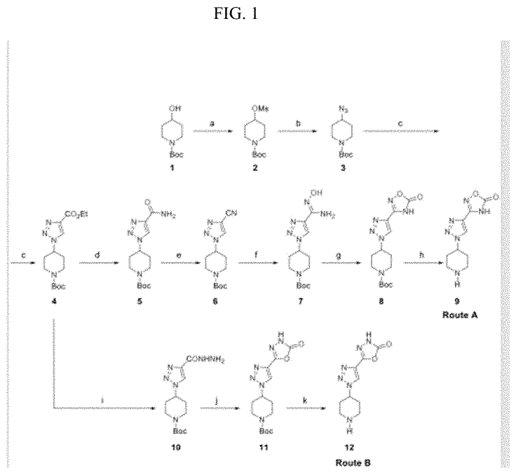

The 1, 2, 3-triazole derivative according to the disclosure can be synthesized by any means known in the art (see, e.g., Sangshetti et al. (2009) Bioorg. Med. Chem. Lett. 19:3564-3567). A useful representative method can be found in EXAMPLE 1 below and in FIG. 1.

Suitable pharmaceutically salts of the 1,2,3-triazole derivatives include acid addition salts with may, for example, be formed by mixing a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid.

Additionally, where the derivatives of the disclosure carry an acidic moiety, suitable pharmaceutically acceptable salts thereof may include alkali metal salts, e.g., sodium or potassium salts, alkaline earth metal salts, e.g., calcium or magnesium salts; and salts formed with suitable organic ligands, e.g. quaternary ammonium salts.

### The Role of Serine Proteases in Hemostasis

The 1, 2,3-triazole derivatives according to the disclosure are useful in part in controlling hemostasis, which maintains blood in a fluid state under physiologic conditions. These derivatives stem abnormal bleeding by affecting the two mechanisms of hemostasis.

The first mechanism of hemostasis comprises two phases. The first phase is characterized by the occurrence of vasoconstriction at the vascular lesion site and platelet aggregation. In the second phase, the fibrin clot is formed due to the action of the different coagulation cascade proteolytic enzymes. This phase and it consists of several steps ending with fibrin polymer formation from fibrinogen hydrolysis due to the action of thrombin. Fibrin polymers are further stabilized by covalent isopeptide bonds formed by factor XIII activated (Factor XIIIa) by thrombin. The mechanical strength of the fibrin gel is useful to impede blood loss when exposed to sheer forces in the circulation. There is a shift in the equilibrium between the formation of soluble fibrin polymers and the assembly of insoluble fibrin fibers. Factor XIIIa lowers the fibrin concentration needed for an insoluble clot to form.

The second mechanism of hemostasis, the fibrinolytic system, is accomplished by localized activation of the plasminogen-plasmin enzyme, whereby it can heal a vascular lesion. Fibrinolysis counteracts the consequences of the coagulation process. The dissolution or solubilization of the fibrin clot at the correct time is needed for the orderly process of wound healing. Fibrinolysis is also required for angiogenesis as recanalization after clot formation. However, excessive local or systemic fibrinolytic activity can result in bleeding, as the weakened plug is dissolved. Conversely, an inadequate fibrinolytic response may retard lysis of a thrombus and contribute to its extension. By a balance of the simultaneous forces of coagulation and platelet aggregation, inhibition of coagulation, pro-fibrinolytic and anti-fibrinolytic reactions, and cellular mechanisms for both coagulation and lysis, the clot is gradually reduced.

Plasmin is a fibrinolytic serine protease that degrades fibrin, and is generated by activation of the zymogen, plasminogen (PLG). PLG is converted to plasmin by two serine protease enzyme plasminogen activators (PA): tissue-type plasminogen activator (tPA); and urokinase-type plasminogen activator (uPA). Secretion of t-PA by endothelial cells may be stimulated by fibrin, by thrombin bound to the thrombus, or by the effects of vessel occlusion, thereby increasing the local concentration of PA. tPA exerts high affinity for fibrin and increased PA activity, whereas, u-PA does not express any interaction with fibrin. tPA converts glu-PLG to the two-chain glu-plasmin. In response to fibrin, endothelial cells are capable of releasing t-PA thereby stimulating the activation of glu-PLG 500-fold, an effect that keeps PLG activation localized to the site of a clot. Once formed, glu-plasmin begins to digest the clot by catalyzing cleavages after selected arginine and lysine residues in the α, β and γ-chains in regions connecting the D- and E-domains of the fibrin protomers.

Hemostasis also reacts to vascular injury to stem blood loss by normal vasoconstriction (the vessel walls closing temporarily), by an abnormal obstruction (such as a plaque), or by coagulation or surgical means (such as ligation).

Abnormal bleeding occurs under certain disease conditions when normal clot formation fails to occur (e.g. hemophilia). Abnormal bleeding may also occur as the result of certain medications prescribed to treat another disorder. In addition, abnormal bleeding also occurs due to physical injuries sustained by otherwise healthy individuals. For example, surgery, dental procedures, accidents, and over-doses of anti-coagulant drugs can result in ruptured vessels and/or organs can result in abnormal bleeding.

In addition, abnormal bleeding may occur due to physically injured ruptured vessels or organs, and has treated by surgical ligation to repair the vessel or organ. However, when surgical ligation of bleeding fails, or is not possible, a number of hemostatic aids have been used. For example, abnormal bleeding can be treated with coagulant drugs such as thrombin, Tissue Factor, Factor VII, and Factor VIIa.

With tissue injury and bleeding, exposed collagen and released tissue factor cause activation of the intrinsic and extrinsic coagulation pathways. Both pathways lead to activation of Factor X which along with activated Factor V forms a complex that cleaves the prothrombin protein into the active thrombin molecule. Thrombin production is the final coagulation step required to cleave fibrinogen into fibrin which provides a hemostatic lattice for platelet aggregation and thrombus formation at the site of injury. Thrombin is often used in conjunction with other hemostatic aids, including absorbable agents (e.g., gelfoam, collagen, and cellulose), and with fibrinogen in fibrin glue.

Factor VII initiates the process of coagulation in conjunction with Tissue Factor. Once bound to Tissue Factor, Factor VII is activated to Factor VIIa by different proteases, including thrombin. Factor VIIa has been used to treat uncontrolled bleeding in hemophilia patients, but there have been safety concerns. Other treatments include protamine sulfate, vitamin K, and plant substances such as leaf of nettle, and water pepper. Antagonists of anti-coagulant drugs, such as protamine sulfate, vitamin K, and inhibitors of fibrinolysis such as aminocaproic acid, contrycal, and aprotinin, have also been used to stem abnormal bleeding.

### Treatment of Bleeding Disorders

1,2,3-triazole derivatives according to the disclosure affect hemostasis by directly inhibiting plasminogen activation and the proteolytic activity of plasmin, t-pa, and u-PA. As such these derivatives are useful in the treatment of bleeding disorders and abnormal bleeding.

The terms “bleeding disorder” and “abnormal bleeding” encompasses disorders and diseases affecting hemostasis and blood coagulation, spontaneous bleeding, cardiac surgery (i.e., cardiopulmonary bypass), liver transplant, following therapeutic thrombolysis, congenital anti-plasmin deficiency, acquired anti-plasmin deficiency, hemophilia A and B, quantitative and qualitative platelet dysfunction, genitourinary bleeding, upper and lower urinary tract, dysfunctional uterine bleeding (essential menorrhagia and menorrhagia associated with intrauterine device), gastrointestinal bleeding (upper by varices, gastritis, ulcers, and lower by inflammatory bowel disease), CCM, cerebral aneurysm, stroke, vasospasm after subarachnoid hemorrhage, spinal cord injury, mucous membrane bleedings for recurrent epistaxis or for excessive bleeding following tonsillectomy, traumatic hyphemia, trauma, general surgery, and orthopedic surgery.

The term “bleeding disorder” as used herein also encompasses physical trauma causing unwanted or uncontrolled bleeding in a subject such as, but not limited to, an accident causing an injury, surgery, dental procedure such as extractions, synovectomy, joint replacement, and in postoperative settings, drugs such as thrombolytic agents, -. The methods are also useful to treat bleeding due to lack of coagulation factors, V, VII, VIII, or IX, or lack of von Willebrand's factor. In addition, the methods according to the disclosure can be used to treat bleeding as the result of administration of an anti-coagulant treatment.

The methods according to the disclosure are useful in the treatment of spontaneous bleeding, cardiac surgery (i.e., cardiopulmonary bypass), liver transplant, following therapeutic thrombolysis, congenital anti-plasmin deficiency, acquired anti-plasmin deficiency, hemophilia A and B, quantitative and qualitative platelet dysfunction, genitourinary bleeding, upper and lower urinary tract, dysfunctional uterine bleeding (essential menorrhagia and menorrhagia associated with intrauterine device), gastrointestinal bleeding (upper by varices, gastritis, ulcers, and lower by inflammatory bowel disease), CCM, cerebral aneurysm, stroke, vasospasm after subarachnoid hemorrhage, spinal cord injury, mucous membrane bleedings for recurrent epistaxis or for excessive bleeding following tonsillectomy, traumatic hyphemia, trauma, general surgery, orthopedic surgery or cancer. The methods are also useful to treat bleeding due to lack of coagulation factors, V, VII, VIII, or IX, or lack of von Willebrand's factor. In addition, the methods according to the disclosure can be used to treat bleeding as the result of administration of an anti-coagulant treatment.

### The Role of Serine Proteases in Cancer

Breakdown of the extracellular matrix is involved in cancer invasion and metastasis. It is accomplished by the concerted action of several proteases, including the serine protease, plasmin, and several matrix metalloproteases. The activity of each of these proteases is regulated by an array of activators, inhibitors and cellular receptors. Thus, the generation of plasmin involves the pro-enzyme, plasminogen, the urokinase type plasminogen activator, uPA, and its pro-enzyme, pro-uPA, the uPA inhibitor PAI-1, the cell surface uPA receptor uPAR, and the plasmin inhibitor a2-anti plasmin.

This system promotes tumor metastasis by several different mechanisms. One of these mechanisms is the uPA and uPAR (urokinase plasminogen activator receptor) system, which initiates the activation of MMPs as well as the conversion of plasminogen to plasmin followed by ECM degradation and reduced cellular interaction.

Plasminogen receptors also play a role in the proliferation and migration of tumor cells in many cancer types and may serve as prognostic and diagnostic markers. They are involved in mediating colocalization of plasminogen and its activators such as uPA and tPA on cell surfaces and decrease the Km for plasminogen activation. Plasminogen receptors are expressed on the cell surface of most tumors and their expression frequently correlates with cancer diagnosis, survival and prognosis. They can trigger multiple specific immune responses in cancer patients, highlighting their role as tumor-associated antigens. Cell surface receptors loaded with plasmin, which are protected from inhibitors, play a key role in cancer progression.

The uPA system, which converts plasminogen into plasmin, plays a key role in cancer invasion and metastasis dissemination by allowing malignant cells to invade the tumor site locally and spread to distant sites. This system includes the serine protease, uPA, membrane-linked receptor uPAR, and two serpin inhibitors, PAI- and PAI-2.

### Treatment of Cancer

By inhibiting tPA, uPA, and plasmin, the 1,2,3-triazole derivatives according to the disclosure can be used to treat cancer and inhibit the metastasis of cancer cells. As such, with the 1,2,3-triazole derivatives according to the disclosure, are useful in treating cancers, such as, but are not limited to, carcinomas, sarcomas, lymphomas, leukemias, germ cell tumors, and blastomas.

### Pharmaceutical Formulations

The pharmaceutical formulations useful in the therapeutic methods according to the disclosure include a therapeutically effective amount of at least one 1,2,3-triazole derivative, and/or a salt thereof.

A “therapeutically effective amount” as used herein refers to that amount which provides a therapeutic and/or prophylactic therapeutic effect for treating a bleeding disorder or trauma resulting in unwanted, uncontrolled bleeding.

In addition, the pharmaceutical formulations according to the disclosure may also comprise more than one 1,2,3-triazole derivative, and/or other known therapeutic agents for stemming bleeding. Such an agent includes, but is not limited to, thrombin, Tissue factor, and/or Factor VIIA. Different combinations of a therapeutically effective amount of at least one derivative according to the disclosure and a therapeutically effective amount of one or more therapeutic anti-bleeding agents can be applied together, e.g. topically.

A “therapeutically effective amount” of a 1,2,3-triazole derivative, or salt thereof, alternatively refers to that amount which treats, kills, and/or controls the growth and/or metastasis of a tumor or cancer cell affecting.

Likewise, the pharmaceutical formulations contain 1,2,3-triazole derivatives according to the disclosure may comprise a therapeutically effective amount of at least one known anti-cancer agent or cancer therapeutic including, but not limited to, alkylating agents (including, but not limited to, cisplatin, chlorambucil, and procarbazine), antimetabolites (including, but not limited to, methotrexate, cytarabine, and gemcitabine), anti-microtubule agents (including, but not limited to, vinblastine and paclitaxel), topoisomerase inhibitors (including, but not limited to, etoposide and doxorubicin) and cytotoxic agents such as, but not limited to, bleomycin.

In the methods according to the disclosure, the pharmaceutical formulations including 1,2,3-triazole derivatives provided herein can be administered alone or in combination with other known therapeutic agents.

The pharmaceutical formulations according to the disclosure further comprise a pharmaceutically acceptable carrier. The term “pharmaceutically acceptable carrier” is to be understood herein as referring to any substance that may, medically, be acceptably administered to a patient, together with a derivative according to the disclosure, and which does not undesirably affect the pharmacological activity thereof; a “pharmaceutically acceptable carrier” may thus be, for example, a pharmaceutically acceptable member(s) comprising of diluents, preservatives, solubilizers, emulsifiers, adjuvant, tonicity modifying agents, buffers as well as any other physiologically acceptable vehicle. These formulations are prepared with the pharmaceutically acceptable carrier in accordance with known techniques, for example, those described in Remington, The Science and Practice of Pharmacy (9th Ed. 1995).

The pharmaceutical formulation may be prepared for injectable use, topical use, oral use, intramuscular or intravenous injection, inhalation use, transdermal use, transmembrane use, and the like.

These formulations are in unit dosage forms such as tablets, pills, capsules, powders, granules, sterile parenteral solutions or suspensions, metered aerosol or liquid sprays, drops, ampoules, auto-injector devices or suppositories; for oral parenteral, intranasal, sublingual topical or rectal administration, or for administration by inhalation or insufflation. Alternatively, the formulations may be presented in a form suitable for one-weekly or once-monthly administration; for example, an insoluble salt of the active compound, such as decanoate salt, may be adapted to provide a depot preparation for intramuscular injection. An erodible polymer containing the active ingredient may be envisaged.

For preparing solid formulations such as tablets, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a 1,2,3-triazole derivative or salt thereof described herein.

These formulations may be homogeneous, i.e., the 1,2,3-triazole derivatives, or salt thereof, is dispersed evenly throughout the formulation so that the formulation may be readily subdivided into equally effective unit dosage forms such as tablets, pills and capsules. A therapeutically effective dosage of the 1,2,3-triazole derivatives according to the disclosure or of another therapeutic which treats a bleeding disorder or cancer may vary from patient to patient, and may depend upon factors such as the age of the patient, the patient's genetics, and the diagnosed condition of the patient, and the route of delivery of the dosage form to the patient. A therapeutically effective dose and frequency of administration of a dosage form may be determined in accordance with routine pharmacological procedures known to those skilled in the art. For example, dosage amounts and frequency of administration may vary or change as a function of time and severity of the disorder. A dosage from about 0.1 mg/kg to 1000 mg/kg, or from about 1 mg/kg to about 100 mg/kg are suitable.

A solid formulation can be subdivided into unit dosage forms of the type described above containing from 0.1 mg to about 500 mg of the active 1,2,3-triazole derivative of the present disclosure. Some useful unit dosage forms contain from 1 to 100 mg, for example 1 mg, 2 mg, 5 mg, 10 mg, 25 mg, 50 mg, or 100 mg, of the derivative. The tablets or pills of the formulation can be coated or otherwise compounded to provide a dosage form affording the advantage of prolonged action. The liquid forms in which the derivatives may be incorporated for administration orally or by injection include aqueous solutions, suitably flavored syrups, aqueous or oil suspensions, and flavored emulsions with edible oils as well as elixirs and similar pharmaceutical vehicles. In the treatment of bleeding episodes or cancer, a suitable dosage level of derivative is about 0.001 mg/kg to about 250 mg/kg per day. The formulations may be administered as a bolus or as a regimen of 1 to about 4 times per day.

Injectable dosage forms may be sterilized in a pharmaceutically acceptable fashion, for example by steam sterilization of an aqueous solution sealed in a vial under an inert gas atmosphere at 120° C. for about 15 minutes to 20 minutes, or by sterile filtration of a solution through a 0.2 μM or smaller pore-size filter, optionally followed by a lyophilization step, or by irradiation of a formulation containing a derivative of the present disclosure by means of emissions from a radionuclide source.

### Activity of Specific 1,2,3-Triazole Derivatives

The enzymatic and/or inhibitory activity of the 1,2,3-triazole derivatives according to the disclosure can be determined by assaying for their fibrinolytic potentials, their serine protease inhibitory activities, and their ability to inhibit cancer growth and migration of cancer cells. These activities can be determined by any assay known in the art, including the following assays.

A. Anti-Fibrinolytic SERPIN) Activity

Fibrinolytic activity in human plasma was determined by a one-step spectrophotometric method (Gidron et al. (1978) J. Clin. Pathol. 31(1):54-57) with minor modifications (see EXAMPLE 2). In this method, fibrin clot formation in anticoagulant citrate dextrose (ACD) plasma samples obtained from healthy volunteers is triggered by tissue factor (TF) and can be quantified by spectrometry as a significant increase in basal plasma absorbance at 340 nm.

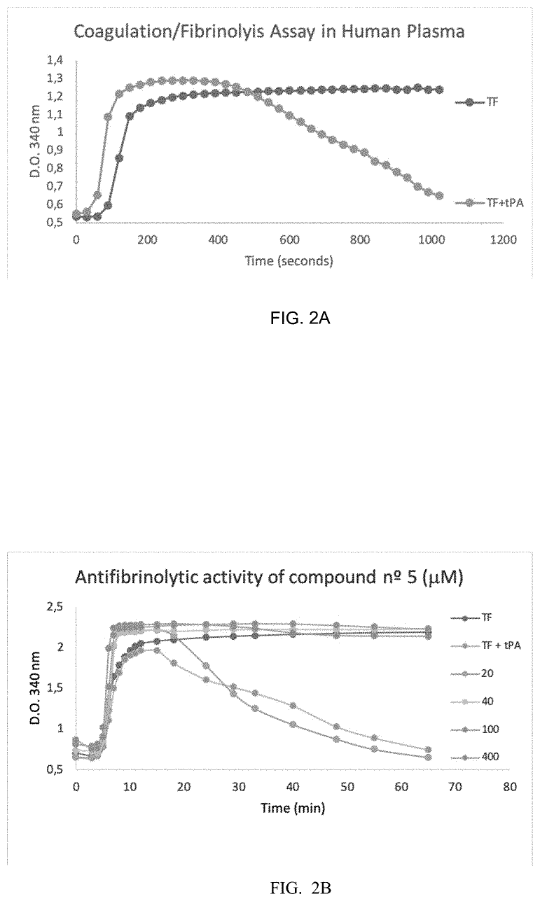

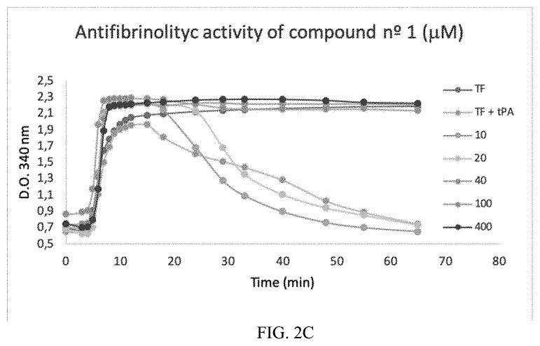

As shown in FIGS. 2A-2D, in the absence of tPA, plasma absorbance remains permanently elevated. In contrast, in the presence of tPA, plasma absorbance returns quickly to basal values, indicating that plasmin was able to lysis completely the formed fibrin clot. The IC50 results (the concentration of derivative at which 50% of total fibrinolysis mediated by tPA is inhibited using this assay in the presence of representative derivatives listed in Table 1 are shown in Table 2 below.

TABLE 2
Anti-fibrinolytic (SERPIN) Activity

| Derivative No. | IC50 μM |
|---|---|
| 1 | 50 |
| 2 | 200 |
| 3 | 180 |
| 4 | 800 |
| 5 | 35 |
| 6 | 375 |
| 7 | 125 |
| 8 | 350 |
| 9 | 500 |
| 10 | 150 |
| 11 | 325 |
| 12 | 25 |
| 13 | 75 |
| 14 | 130 |
| 15 | 24 |
| 16 | 58 |
| 17 | 320 |
| 18 | 250 |
| 19 | 375 |
| 20 | 15 |
| 21 | 35 |
| 22 | 50 |
| 23 | 35 |
| 24 | 315 |
| 25 | 205 |
| 26 | 153 |
| 27 | 189 |
| 28 | 23 |
| 29 | 67 |
| 30 | 370 |
| 31 | 74 |
| 32 | 35 |
| 33 | 29 |
| 34 | 101 |
| 35 | 290 |
| 36 | 119 |

These results show that 1,2,3-triazole derivatives according to the disclosure have anti-fibrinolytic activity of varying degrees.

B. Cancer Inhibition

The ability of 1,2,3-triazole derivatives according to the disclosure to inhibit cancer cell migration and proliferation in vitro can be measured, e.g., by a wound healing assay (Rodriguez et al., in Cell Migration: Developmental Methods and Protocols, Web et al., Guan ed. Humana Press 294, 23-29). This assay is based on the observation that, upon the creation of an artificial gap on a confluent cancer cell monolayer, the cells on the edge of the created gap will start migrating and proliferating until new cell-cell contacts are established (EXAMPLE 3).

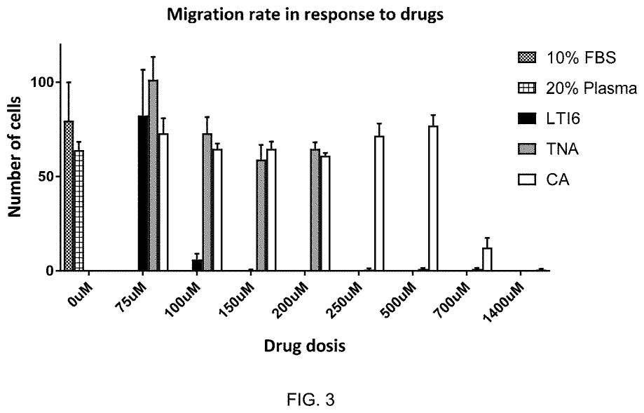

As shown in FIG. 3, and also below in Table 3, where increasing amounts of representative derivatives (Table 1) were used to attain a dose-response curve, 1,2,3-triazole derivatives according to the disclosure significantly inhibit the proliferation and migration of cancer cells.

TABLE 3
Anti-Cancer Activity (Inhibition of Cancer Cell Migration)

| Derivative No. | IC50 μM |
|---|---|
| 1 | 120 |
| 2 | 350 |
| 3 | 230 |
| 4 | 1200 |
| 5 | 90 |
| 6 | 650 |
| 7 | 270 |
| 8 | 500 |
| 9 | 820 |
| 10 | 250 |
| 11 | 510 |
| 12 | 65 |
| 13 | 125 |
| 14 | 250 |
| 15 | 104 |
| 16 | 250 |
| 17 | 650 |
| 18 | 345 |
| 19 | 610 |
| 20 | 87 |
| 21 | 100 |
| 22 | 120 |
| 23 | 89 |
| 24 | 620 |
| 25 | 420 |
| 26 | 258 |
| 27 | 310 |
| 28 | 69 |
| 29 | 129 |
| 30 | 520 |
| 31 | 190 |
| 32 | 101 |
| 33 | 82 |
| 34 | 220 |
| 35 | 450 |
| 36 | 278 |

The ability of 1,2,3-triazole derivatives according to the disclosure to kill cancer cells can be measured, e.g., by Trypan Blue Exclusion Assay which measured cell death by starvation due to the presence of the derivative (see EXAMPLE 4). The results obtained using this assay with some representative 1,2,3-triazole derivatives according to the disclosure are shown below in Table 4. These results show the IC50 of some representative derivatives according to the invention (which is the concentration of derivative causing greater than 90% of the cells to die.

TABLE 4
Anti-Cancer Activity (Trypan Blue Exclusion Assay)

| Derivative No. | IC50 μM |
|---|---|
| 1 | 250 |
| 2 | 550 |
| 3 | 410 |
| 4 | 2100 |
| 5 | 150 |
| 6 | 800 |
| 7 | 415 |
| 8 | 750 |
| 9 | 1100 |
| 10 | 370 |
| 11 | 680 |
| 12 | 110 |
| 13 | 170 |
| 14 | 450 |
| 15 | 815 |
| 16 | 430 |
| 17 | 1350 |
| 18 | 550 |
| 19 | 880 |
| 20 | 210 |
| 21 | 150 |
| 22 | 210 |
| 23 | 210 |
| 24 | 810 |
| 25 | 610 |
| 26 | 470 |
| 27 | 480 |
| 28 | 140 |
| 29 | 218 |
| 30 | 710 |
| 31 | 310 |
| 32 | 101 |
| 33 | 184 |
| 34 | 350 |
| 35 | 670 |
| 36 | 415 |

Reference will now be made to specific examples illustrating the disclosure. It is to be understood that the examples are provided to illustrate exemplary embodiments and that no limitation to the scope of the disclosure is intended thereby.

### EXAMPLES

### Example 1

### Synthesis of 1,2,3-Triazole Derivatives

The synthesis of representative, 2,3-triazole derivatives according to the disclosure is summarized in the synthetic scheme shown in FIG. 1 and described below. The compound numbers recited below correspond to those compounds set forth in this example and not to the derivative numbers listed in Table 1 supra.

All reagents were purchased from Sigma Aldrich or FluoroChem and were used without further purification. The progress of all reactions was monitored on Merck precoated silica gel plates (with fluorescence indicator UV2S4) using ethyl acetate/cyclohexane as solvent system. Column chromatography was performed with Merck silica gel 60 (230-400 mesh particle size). Proton (¹H) and carbon (¹³C) NMR spectra were recorded on a Varian 400 (400 MHz for ¹H; 100.6 MHz for ¹³C) using chloroform-d or DMSO-d₆ as solvent. Chemical shifts are given in parts per million (ppm) (δ relative to residual solvent peak for ¹H and ¹³C. Elemental analysis was performed on an EA3000 elemental analyzer.

The 1,2,4-oxadiazole derivative (compound 9) was synthesized following the protocol described by Sangshetti et al. (Bioorg. Med. Chem. Lett. (2009) 19:3564-3567). The synthesis for the 1,3,4-oxadiazole derivative (compound 12) was partially based on the work by Jansen et al. (J. Med. Chem. (2008) https://doi.org/10.1021/jm701562x).

Physical appearance, yield, and structural determination results for all intermediates and final products (FIG. 1 of synthetic pathway) are listed below. The complete methodology is included for the synthesis of compound 12 starting from compound 4.

### tert-butyl 4-((methylsulfonyl)oxy)piperidine-1-carboxylate (2)

Pale yellow solid; yield 97%. ¹H NMR (400 MHz, CDCl₃), δ (ppm): 4.84-4.90 (m, 1H), 3.66-3.72 (m, 2H), 3.25-3.32 (m, 2H), 3.02 (s, 3H), 1.91-1.99 (m, 2H), 1.76-1.84 (m, 2H), 1.44 (s, 9H).

### tert-butyl 4-azidopiperidine-1-carboxylate (3)

Yellow oil; yield 87%. ¹H NMR (400 MHz, CDCl₃), δ (ppm): 3.81-3.76 (m, 2H), 3.51-3.56 (m, 1H), 3.02-3.09 (m, 2H), 1.79-1.86 (m, 2H), 1.49-1.56 (m, 2H), 1.44 (s, 9H).

### tert-butyl 4-(4-(ethoxycarbonyl)-1H-1,2,3-triazol-1-yl)piperidine-1-carboxylate (4)

Pale yellow oil; yield 84%. ¹H NMR (400 MHz, CDCl₃), δ (ppm): 8.08 (s, 1H), 4.62-4.70 (m, 1H), 4.40 (q, J=7.1 Hz, 2H), 4.23-4.31 (m, 2H), 2.89-2.93 (m, 2H), 2.18-2.23 (m, 2H), 1.88-1.98 (m, 2H), 1.46 (s, 9H), 1.39 (t, J=7.1 Hz, 3H).

### tert-butyl 4-(4-carbamoyl-1H-1,2,3-triazol-1-yl)piperidine-1-carboxylate (5)

White solid; yield 91%. ¹H NMR (400 MHz, CDCl₃), δ (ppm): 8.10 (s, 1H), 7.00 (s, 1H), 5.70 (s, 1H), 4.62-4.70 (m, 1H), 4.24-4.31 (m, 2H), 2.89-3.01 (m, 2H), 2.19-2.23 (m, 2H), 1.88-1.99 (m, 2H), 1.44 (s, 9H).

### tert-butyl 4-(4-cyano-1H-1,2,3-triazol-1-yl)piperidine-1-carboxylate (6)

Brown oil; yield 97%. ¹H NMR (400 MHz, CDCl₃), δ (ppm): 8.12 (s, 1H), 4.61-4.72 (m, JH), 4.25-4.30 (m, 2H), 2.89-3.03 (m, 2H), 2.20-2.26 (m, 2H), 1.92-2.04 (m, 2H), 1.48 (s, 9H).

### tert-butyl (Z)-4-(4-(N′-hydroxycarbamimidoyl)-1H-1,2,3-triazol-1-yl)piperidine-1-carboxylate (7)

White solid; yield 23%. ¹H NMR (400 MHz, d₆-DMSO), δ (ppm): 9.50 (s, 1H), 8.35 (s, 1H), 5.71 (s, 2H), 4.69-4.77 (m, 1H), 4.03-4.07 (m, 2H), 2.95 (s, 2H), 2.05-2.09 (m, 2H), 1.82-1.93 (m, 2H), 1.42 (s, 9H).

### tert-butyl 4-(4-(5-oxo-4,5-dihydro-1,2,4-oxadiazol-3-yl)-1H-1,2,3-triazol-1-yl)piperidine-1-carboxylate (8)

White solid; yield 6%. ¹H NMR (400 MHz, d₆-DMSO), δ (ppm): 8.57 (s, 1H), 6.92 (s, 1H), 4.71-4.77 (m, 1H), 4.00-4.03 (m, 2H), 2.82-3.01 (m, 2H), 2.03-2.08 (m, 2H), 1.81-1.89 (m, 2H), 1.38 (s, 9H). ¹³C NMR (100.6 MHz, d⁶-DMSO), δ (ppm): 159.6, 154.2, 151.9, 150.6, 139.8, 79.4, 57.5, 42.9, 31.6, 28.4. HRMS (ESI-FIA-TOF): m/z calculated for C₁₄H₂₁N₆O₄ 337.1624, found 337.1619.

### hydrochloride of 3-(1-(piperidin-4-yl)-1H-1,2,3-triazol-4-yl)-1,2,4-oxadiazol-5(4H)-one (9)

White solid; yield 90%. ¹H NMR (400 MHz, d₆-DMSO), δ (ppm): 13.28 (s, 1H), 9.10 (s, 1H), 8.90 (s, 1H), 8.89 (s, 1H), 4.92 (m, 1H), 3.32 (m, 2H), 3.09 (m, 2H), 2.33-2.24 (m, 4H). ¹³C NMR (100.6 MHz, d⁶-DMSO), δ (ppm): 159.4, 151.5, 131.9, 124.4, 55.1, 41.7, 30.6, 28.3.

### tert-butyl 4-(4-(hydrazinecarbonyl)-1H-1,2,3-triazol-1-yl)piperidine-1-carboxylate (10)

Tert-butyl 4-(4-(ethoxycarbonyl)-1H-1,2,3-triazol-1-yl)piperidine-1-carboxylate (1 g, 3.1 mmol) and hydrazine hydrate (0.5 g) in 20 mL of n-butanol were refluxed for 3 h. Then, the solvent was removed by evaporation under vacuum. The residue was treated with dichloromethane and washed with water. The organic phase was dried (MgSO4) and the solvent removed under reduced pressure. The resulting solid (0.93 g) was washed with cold ethanol.

Pale yellow solid; yield 97%. ¹H NMR (400 MHz, CDCl₃), δ (ppm): 8.08 (s, 1H), 4.63 (tt, J=11.6, 3.6 Hz, 1H), 4.27 (s, 2H), 4.03 (s, 1H), 2.94 (t, J=13.0 Hz, 2H), 2.21 (d, J=12.8 Hz, 2H), 1.94 (qd, J=12.0, 4.4 Hz, 2H), 1.47 (d, J=0.6 Hz, 9H).

### tert-butyl 4-(4-(5-oxo-4,5-dihydro-1,3,4-oxadiazol-2-yl)-1H-1,2,3-triazol-1-yl)piperidine-1-carboxylate (11)

To tert-butyl 4-(4-(hydrazinecarbonyl)-1H-1,2,3-triazol-1-yl)piperidine-1-carboxylate (10) (0.5 g, 1.6 mmol) in a mixture of 20 mL of THF and 2 mL of DMF were added subsequently N,N′-carbonyldiimidazole (CDI) (0.4 g, 2.5 mmol) and triethylamine (0.32 g, 3 mmol). After refluxing for 15 h, the solvent was removed by evaporation under vacuum. The residue was treated with dichloromethane and washed with water. The organic phase was dried (MgSO4) and the solvent removed under reduced pressure. Chromatography yielded 0.5 g of tert-butyl 4-(4-(5-oxo-4,5-dihydro-1,3,4-oxadiazol-2-yl)-1H-1,2,3-triazol-1-yl)piperidine-1-carboxylate.

White solid; yield 94%. ¹H NMR (400 MHz, CDCl₃), δ (ppm): 9.52 (s, 1H), 8.05 (s, 1H), 4.70 (tt, J=11.7, 4.1 Hz, 1H), 4.29 (d, J=11.9 Hz, 2H), 2.96 (t, J=12.7 Hz, 2H), 2.38-2.17 (m, 2H), 2.17-1.88 (m, 2H), 1.47 (s, 9H). ¹³C NMR (100.6 MHz, d⁶-DMSO), δ (ppm): 154.5, 154.1, 148.7, 133.6, 124.1, 79.4, 58.2, 42.2, 32.0, 28.50. HRMS (ESI-FIA-TOF): m/z calculated for C₁₄H₂₀N₆NaO₄ 359.1444, found 359.1438.

### 5-(1-(piperidin-4-yl)-1H-1,2,3-triazol-4-yl)-1,3,4-oxadiazol-2(3H)-one (12) hydrochloride

A mixture of tert-butyl 4-(4-(5-oxo-4,5-dihydro-1,3,4-oxadiazol-2-yl)-1H-1,2,3-triazol-1-yl)piperidine-1-carboxylate (11) (100 mg, 0.3 mmol) and 4N HCl in dioxane (2 mL) was stirred at RT for 2 h. The solvent was removed in vacuo and the resulting yellow solid was triturated with EtOAc to provide 74 mg of the hydrochloride of 5-(1-(piperidin-4-yl)-1H-1,2,3-triazol-4-yl)-1,3,4-oxadiazol-2(3H)-one. White solid; yield 91%. ¹H NMR (400 MHz, d⁶-DMSO), δ (ppm): 13.24 (s, 1H), 9.26 (s, 1H), 9.10 (s, 1H), 8.24 (s, 1H), 5.28-5.02 (m, 1H), 3.38 (d, J=12.7 Hz, 2H), 3.05 (q, J=11.7 Hz, 2H), 2.40-1.99 (m, 4H). ¹³C NMR (100.6 MHz, d⁶-DMSO), δ (ppm): 154.0, 148.1, 133.1, 124.2, 54.9, 41.7, 28.2.

### Example 2

### Fibrinolytic Assays

Fibrinolytic activity in human plasmas in the presence of derivatives according to the disclosure was determined by a one-step spectrophotometric method.

Fibrin clot formation in the anticoagulant acid citrate dextrose (ACD)-treated plasma obtained from healthy volunteers is triggered by tissue factor (TF) (Thromborel S, Siemens Healthineers) and quantifies by spectrophotometry at 340 nm (using a Molecular Devices SpectraMax® M2e Multimode Microplate Reader). When recombinant human Tissue Plasminogen Activator (tPA, final concentration of 5.2 μg/ml) is simultaneously added, fibrinolysis of the clot can be determined.

Fresh blood was extracted from healthy volunteers. ACD, to reach a 10% concentration, was instantly added to the extracted blood, to prevent any unwanted coagulation. After that, blood was centrifuged for 15 min at 1,000 g. Plasma was clearly separated from red blood cells. 3 mL aliquots of aspirated plasma were immediately frozen at −20° C.

4 mL of ddH2O was added to a vial of Thromborel S and mixed thoroughly for 2 min. and left at 4° C. 500 μL TF aliquots were used for each experiment.

Representative derivatives of the present disclosure (Nos. 1, 5, and 7) were diluted in DMSO (those with Boc) or ddH2O (those with HCl) to a final concentration of 10 mM. A solution containing 1 M Tris HCl, pH 7.5 (Fisher BioReagent) (Buffer A), 0.110 g of CaCl₂) in 10 mL of ddH2O (Buffer B), and tPA (Abcam, 1.3 mg/mL added into the diluted derivative solution, final concentration in serum was 5.2 μg/mL), and the derivative was prepared in a total volume of 75 μL and added to untreated, flat, clear Costar® 96-well plates in triplicate. Absorbance at 340 nm at 37° C. for 30 min was recorded every 15 sec. The plates were shaken every 3 sec in between readings. Controls for basal plasma absorbance, basal coagulation absorbance and basal fibrinolytic activity absorbance were performed in every assay.

FIG. 2A show the results a control experiment displaying the ability of TF to cause fibrin clot formation in the absence and presence of t-PA. TF was able to cause fibrin clot formation evaluated as a significant increase of the basal plasma absorbance at 340 nm. In the absence of t-PA, plasma absorbance remained permanently elevated, in contrast, in the presence of t-PA, plasma absorbance returned quickly to basal values, indicating that plasmin was able to completely lyse the formed fibrin clot.

FIGS. 2B, 2C, and 2D show the results of clot formation experiments done in the presence of different concentrations of several representative 1,2,3 derivative according to the disclosure. FIG. 2B shows the antifibrinolytic effect produced by Derivative 5, FIG. 2C the effect produced by Derivative 1, and FIG. 2D shows the effect of Derivative 7. All derivatives were able to completely inhibit fibrinolysis mediated by tPA.

As shown in Table 2, all the 1,2,3-triazole derivatives displayed inhibitory fibrinolytic activity against plasminogen activation by tPA in varying degrees.

### Example 3

### Cancer Migration (Wound Healing) Assay

Non-Small Lung Cancer Cells (NSLCs) migration rate was assessed in an in vitro wound healing assay. This assay is based on the observation that, upon the creation of an artificial gap on a confluent cell monolayer, the cells on the edge of the created gap will start migrating and proliferating until new cell-cell contacts are established.

A confluent cell layer is a prerequisite for starting this assay. Gap formation is done manually with a scrapper and cell proliferation and migration is determined recording a time-lapse video for 20 hr. with a time interval of 30 min. The microscopic pictures are manually analyzed for obtaining information about the proliferation and migration characteristics of the cultured cells and the image analysis detects the cell covered area. Plotting the cell covered area against the time showed the process of gap closure, the proliferation and migration of cancer cells is determined.

NSLCs were cultured in Eagle's Minimum Essential Medium (ATCC-formulated medium, Catalog No. 30-2003) containing fetal bovine serum at 10% during several periods. Wound healing assays with supplemented medium containing 1,2,3-triazole derivatives were performed to evaluate the inhibitory effect on proliferation and migration of cancer cells.

In order to simulate as close as possible the in vivo cancer microenvironment, human plasma and calcium were added to the cell culture system to provide the hemostatic factors normally present in the cancer stroma. The previous day, cells were seeded with 10% FBS at a number that would provide 90% confluence the next day. On Day 0, a scratch (“a wound”) was performed in the middle of the well and the 10% FBS medium was replaced with 20% plasma, 20 mM calcium medium, with or without Derivative 1. 10% FBS medium was used as control in these experiments. “Wound healing” or closure of the scratch was assessed over a period of 4 days, and cell number in the wound area was quantified for each condition. Increasing amount of Derivative 1 (LTI6) was used to attain a dose-response curve.

The cell function effect of Derivative 1 was compared with Tranexamic Acid (TNA) and Caproic Acid (CA), commercially available and widely used anti-metastasis drugs.

As shown in FIG. 3, cell migration was completely impaired in the presence of 100 μM Derivative 1 (LTI6), 250 μM TNA, and 1400 μM CA. The dose response curve provided the following IC50: LTI6=96.11 μM; TNA=438.12 μM, CA=1344.84 μM. Therefore, Derivative 1 is a more potent drug than TNA (>4×) and CA (>10×) in the inhibition of migration of cancer cells.

Other derivatives according to the disclosure were also tested, and their IC50s in this test are shown above in Table 2. These derivatives also impaired cell migration.

### Example 4

### Trypan Blue Exclusion Test of Cancer Cell Viability

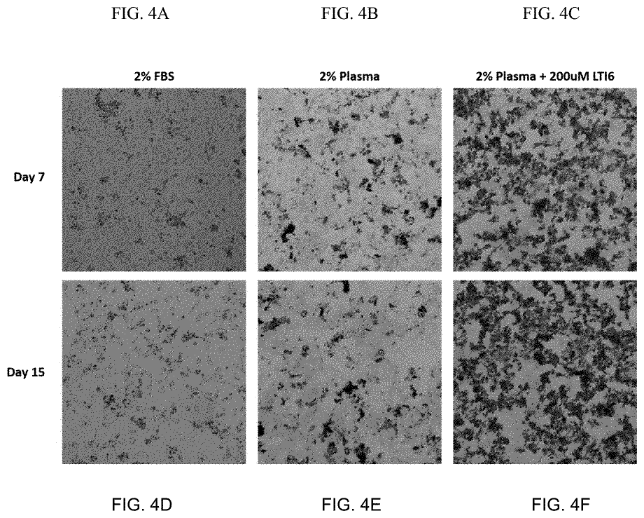

The effect of Derivative 1 on cancer cell viability was determined using the Trypan Blue Exclusion Test (Stober (2001) (Curr. Protoc. Immunol., May; Appendix 3:Appendix 3B. doi: 10.1002/0471142735.ima03bs21). The viability of Non-Small Lung Cancer Cells (NSLCs) was assessed over a period of 15 days after the promotion of coagulation with or without 200 μM of 1,2,3-triazole derivative 1 and with or without 2% human plasma. Fresh 2% FBS medium corresponding to each condition was added every 3 days. Cell death was analyzed using Trypan blue staining. 2% FBS medium was used as control. Some representative results are shown in FIGS. 4A-4F.

After 7 days, cells in 2% FBS appear to be healthy although not proliferating (FIG. 4A). Under the 2% Plasma conditions, a higher number of dead cells was visible (FIG. 4B), but this effect was even more significant in the presence of Derivative 1 at a concentration of 200 μM (FIG. 4C). After 15 days in the presence of Derivative 1, increased staining due to the increase in detached cells indicative of increased cell death was observed (FIG. 4F), relative to the number of attached cells, in both the 2% FBS (FIG. 4D) and 2% Plasma conditions (FIG. 4E) (which was likely due to lack of nutrients and removal of old medium. These results indicate that persistent coagulation promoted by Derivative 1 leads to increased cell starvation, since cells are trapped in the fibrin mesh and nutrition is not able to reach them.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A compound of Formula I: 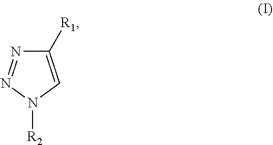
or a pharmaceutically acceptable salt thereof, wherein:
R₁ is selected from the group consisting of hydrogen, 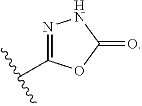 [SMILES: *c1n[nH]c(=O)o1]
A is independently, at each occurrence, O or NR₄;
R₃ is independently, at each occurrence, NH₂, OH, or NHNH₂;
R₄ is independently, at each occurrence, H or OH;
R₂ is selected from the group consisting of 3-6 membered cycloalkyl, 3-6 membered heterocycloalkyl or C₁-C₆ alkylamine, wherein cycloalkyl, heterocycloalkyl, and alkyl are optionally substituted with one or two R₅; and
R₅ is independently, at each occurrence, selected from the group consisting of NH₂, OH, SH, and halo.

2. The compound of claim 1, wherein R₁ is

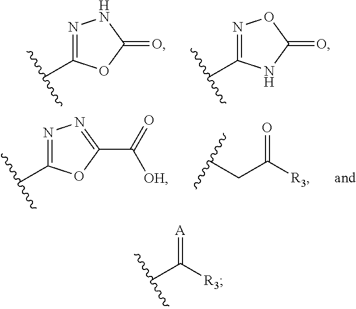

3. The compound of claim 1, wherein R₁ is

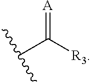

4. The compound of claim 1 or 3, wherein R₁ is

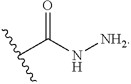 [SMILES: *C(=O)NN]

5. The compound of claim 1 or 3, wherein R₁ is

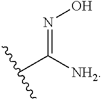 [SMILES: */C(N)=N/O]

6. The compound of claim 1 or 3, wherein R₁ is

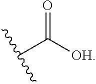 [SMILES: *C(=O)O]

7. The compound of claim 1, wherein R₁ is

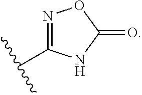 [SMILES: *c1noc(=O)[nH]1]

8. The compound of claim 1, wherein R₁ is

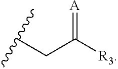

9. The compound of claim 1 or 8, wherein R₁ is

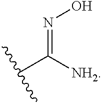 [SMILES: */C(N)=N/O]

10. The compound of claim 1, wherein R₁ is

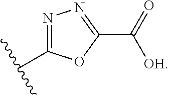 [SMILES: *c1nnc(C(=O)O)o1]

11. The compound of any one of claims 1-10, wherein R₂ is selected from the group consisting of

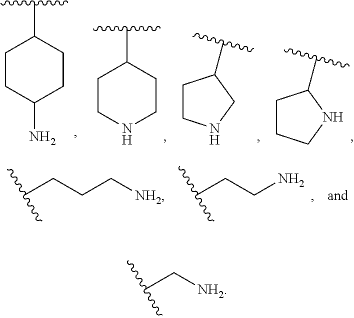

12. The compound of any one of claims 1-11, wherein R₂ is

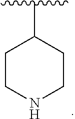 [SMILES: *C1CCNCC1]

13. The compound of any one of claims 1-11, wherein R₂ is

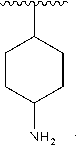 [SMILES: *C1CCC(N)CC1 ; "."]

14. The compound of any one of claims 1-11, wherein R₂ is

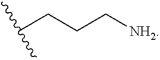 [SMILES: *CCCN]

15. The compound of any one of claims 1-11, wherein R₂ is

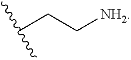 [SMILES: *CCN]

16. The compound of claim 1, wherein the compound of Formula I is selected from the group consisting of

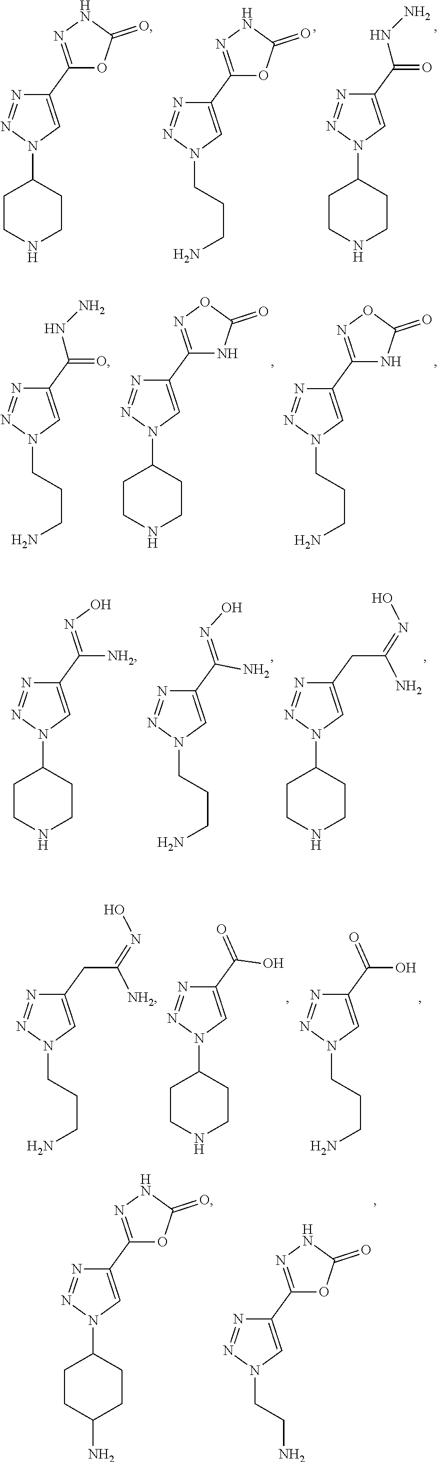

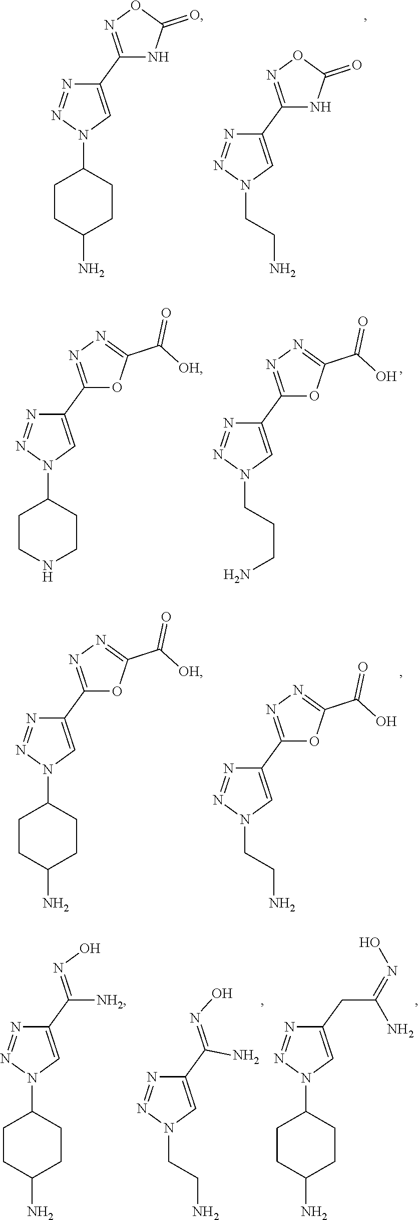

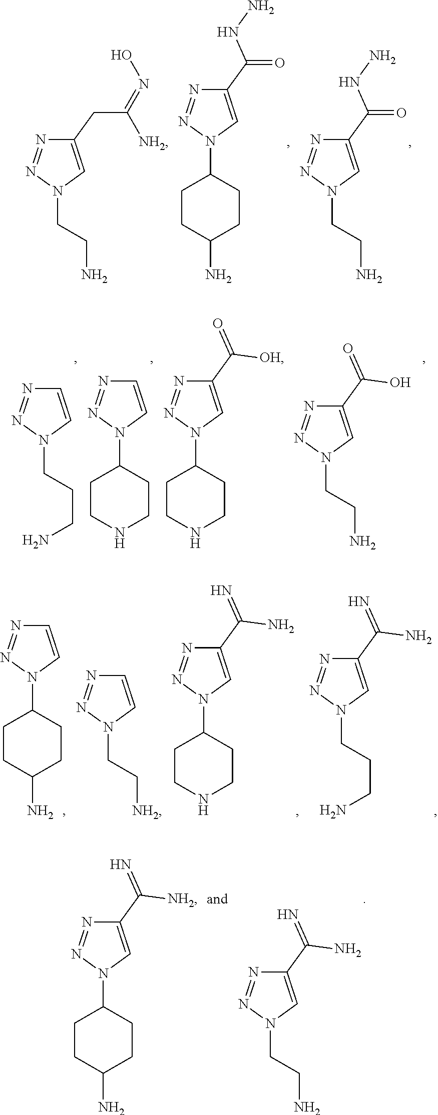

17. A pharmaceutical formulation comprising a compound of claim 1 and a pharmaceutically acceptable carrier.

18. A method of treating bleeding in a subject, comprising administering to the subject a therapeutically effective amount of the formulation of claim 17.

19. A method of treating cancer or metastasis in a subject, comprising administering to the subject a therapeutically effective amount of the formulation of claim 17.

20. A method of inhibiting the serine protease activity of an enzyme selected from the group consisting of tissue plasminogen activator, urokinase plasminogen activator, or plasmin, comprising contacting the enzyme with the compound of claim 1.
